(19) United States
(12) Patent Application Publication
(10) Pub. No.: US 2025/0051440 A1
(43) Pub. Date: Feb. 13, 2025

(54) DEPLETION OF NK CELLS FOR THE TREATMENT OF ADVERSE POST-ISCHEMIC CARDIAC REMODELING

(71) Applicants: INSTITUT NATIONAL DE LA SANTÉ ET DE LA RECHERCHE MÉDICALE, Paris (FR); SORBONNE UNIVERSITÉ, Paris (FR); UNIVERSITÉ PARIS CITÉ, Paris (FR); ASSISTANCE PUBLIQUE-HÔPITAUX DE PARIS (APHP), Paris (FR)

(72) Inventors: Hafid AIT-OUFELLA, Paris (FR); Vincent DUVAL, Paris (FR)

(21) Appl. No.: 18/718,492

(22) PCT Filed: Dec. 13, 2022
(86) PCT No.: PCT/EP2022/085715

§ 371 Date: Jun. 11, 2024

(30) Foreign Application Priority Data
Dec. 14, 2021 (EP) 21306764.8

(51) Int. Cl. C07K 16/28; A61K 39/00; A61K 47/68; A61P 9/04

(52) U.S. Cl. CPC C07K 16/2803; A61K 47/6849; A61P 9/04; A61K 2039/505; C07K 2317/31; C07K 2317/732

(57) ABSTRACT

Myocardial infarction (MI), the most prevalent manifestation of cardiovascular diseases, is associated with high mortality and morbidity. In particular, long term effects of ischemia-related cardiac damage continue to be a clinical and social burden, due to increased risk of arrhythmias, heart failure and repetitive hospitalizations. Therefore, there is a medical need for the development of therapeutic approaches targeting pathophysiological pathways involved in post-ischemic cardiac remodeling. Now, the inventors obtained several evidences confirming that NK cells promote deleterious post-ischemic cardiac remodeling. In particular, the inventors showed that i) NK cells are recruited in the ischemic heart tissue in mouse, ii) NK cells are detected in human ischemic heart tissue, and iii) NK deficiency protects against deleterious post-ischemic cardiac remodeling, and iiii) NK cell depletion using monoclonal antibody in mice protects against deleterious post-ischemic cardiac remodelling and consecutive ischemic heart failure.

### FIELD OF THE INVENTION

The present invention is in the field of medicine, in particular cardiology.

### BACKGROUND OF THE INVENTION

Myocardial infarction (MI), the most prevalent manifestation of cardiovascular diseases, is associated with high mortality and morbidity [1]. Nevertheless, considerable advances have been achieved in the early management of acute coronary thrombotic occlusion, including rapid mechanical restauration of coronary artery blood flow and anti-platelet therapies [2]. A marked decline in early mortality of patients with MI has been observed over the last decades [3]. However, long term effects of ischemia-related cardiac damage continue to be a clinical and social burden, due to increased risk of arrhythmias, heart failure and repetitive hospitalizations [4]. Therefore, more efforts have to be deployed towards the development of therapeutic approaches targeting pathophysiological pathways involved in post-ischemic cardiac remodeling.

Although extensive knowledge has been acquired over the past few years on the initiation of the inflammatory response after MI, only recently anti-IL1b has been shown effective as a specific therapeutic strategy targeting the immuno-inflammatory response in atherosclerosis [5] and, so far, no immune-modulatory medical therapy has been approved for the treatment or prevention of adverse remodeling in acute MI. This may be due, at least in part, to the fact that little is known about the specific roles of the various components of the immune response in the process of post-ischemic myocardial remodeling. Therefore, the specific immune cell subsets that critically orchestrate the various phases of the immune response to ischemic injury need to be better characterized.

Natural Killer (NK) cells comprise the largest subset of the innate lymphoid cell family [6]. NK cells are morphologically lymphocytic in nature, but lack the somatic rearrangement of antigen receptors found in the more classical T-cells and B-cells of the adaptive immune system. NK cells are responsive to multiple immune signals required for defense against pathogens, and are necessary for the formation of lymphoid organs [7]. Additionally, NK cells play a major role in both repairing damaged tissue and maintaining tissue homeostasis [8]. NK cells share many common features with ILC1 cells [9]. In human, in the context of acute MI, NK population drops in the blood following coronary reperfusion suggesting a recruitment within the ischemic tissue [10]. However, little is known about the role of NK cells in post-ischemic cardiac remodeling. In rats, NK cell infiltration reaches a peak 7 days after a cardiac ischemia [11]. In experimental stroke, their accumulation in infarcted brain has been shown to the detrimental to disease outcomes [12]. Conversely, in hepatic ischemia-reperfusion injury models, NK cells are protective through ligation of tumor necrosis factor-related apoptosis-inducing ligand [13].

### SUMMARY OF THE INVENTION

The present invention is defined by the claims. In particular, the present invention relates to a method of treating adverse post-ischemic cardiac remodeling in a patient who experienced a myocardial infarction comprising administering to the patient a therapeutically effective amount of an agent that depletes NK cells.

### DETAILED DESCRIPTION OF THE INVENTION

The inventors obtained several evidences confirming that NK cells promote deleterious post-ischemic cardiac remodeling. In particular, the inventors showed that i) NK cells are recruited in the ischemic heart tissue in mouse, ii) NK cells are detected in human ischemic heart tissue iii) NK deficiency protects against deleterious post-ischemic cardiac remodelling and iiii) NK cell depletion using monoclonal antibody in mice protects against deleterious post-ischemic cardiac remodelling and consecutive ischemic heart failure.

The first object of the present invention relates to a method of treating adverse post-ischemic cardiac remodeling in a patient who experienced a myocardial infarction comprising administering to the patient a therapeutically effective amount of an agent that depletes NK cells.

As used herein, the term “subject”, “individual” or “patient” is used interchangeably and refers to any subject for whom diagnosis, treatment, or therapy is desired, particularly humans.

Other subjects may include cattle, dogs, cats, guinea pigs, rabbits, rats, mice, horses, and the like. In some preferred embodiments, the subject is a human.

As used herein, the term “myocardial infarction” has its general meaning in the art and relates to the irreversible necrosis of the myocardium as a result of prolonged ischemia due to coronary thrombosis, i.e. the development of a clot in a major blood vessel serving the heart.

As used herein, the term “adverse post-ischemic cardiac remodeling” has its general meaning in the art and refers to the prominent changes that occur after myocardial infarction and that could be deleterious for the cardiac function. Cardiac remodeling involves molecular, cellular, and interstitial changes that manifest clinically as changes in size, shape, and function of the heart which occur after myocardial infarction. For instance, ventricular remodeling involves progressive enlargement of the ventricle with depression of ventricular function. Myocyte function in the myocardium remote from the initial myocardial infarction becomes depressed.

In particular, adverse post-ischemic cardiac remodeling includes arrhythmias, cardiac dilation (assessed by left ventricular end diastolic volume indexed on body surface area or LVEDVi) and cardiac dysfunction (left ventricular ejection fraction or EF). Typically, adverse post-ischemic cardiac remodeling is defined as a >20% increase in left ventricular end-diastolic volume (LVEDV) at 6 months as compared to the initial evaluation.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In particular, the method of the present invention is suitable for protecting against or reducing damage to the myocardium after a myocardial infarction, after, during or prior to ischemic reperfusion. More particularly, the method of the present invention is particularly suitable for reducing post ischemic left ventricular remodeling. Even more particularly, the method of the invention is suitable for increasing the left ventricle ejection fraction (LVEF), and/or for inhibiting left ventricle enlargement, and/or for reducing left ventricle end systolic volume, and/or reducing left ventricle end diastolic volume, and/or for ameliorating left ventricle dysfunction, and/or for improving myocardial contractibility.

The method of the present invention is also suitable for preventing heart failure in a patient who experienced a myocardial infarction.

As used herein, the term “heart failure” or “HF” has its general meaning in the art and embraces congestive heart failure and/or chronic heart failure. Functional classification of heart failure is generally done by the New York Heart Association Functional Classification (Criteria Committee, New York Heart Association. Diseases of the heart and blood vessels.

Nomenclature and criteria for diagnosis, 6th ed. Boston: Little, Brown and co, 1964; 114). This classification stages the severity of heart failure into 4 classes (I-IV). The classes (I-IV) are: Class I: no limitation is experienced in any activities; there are no symptoms from ordinary activities; Class II: slight, mild limitation of activity; the patient is comfortable at rest or with mild exertion; Class III: marked limitation of any activity; the patient is comfortable only at rest; Class IV: any physical activity brings on discomfort and symptoms occur at rest.

In some embodiments, the depleting agent of the present invention is administered to a patient having one or more signs or symptoms of acute myocardial infarction injury. In some embodiments, the patient has one or more signs or symptoms of myocardial infarction, such as chest pain described as a pressure sensation, fullness, or squeezing in the mid portion of the thorax; radiation of chest pain into the jaw or teeth, shoulder, arm, and/or back; dyspnea or shortness of breath; epigastric discomfort with or without nausea and vomiting; and diaphoresis or sweating.

In particular embodiments, the patient is administered with the depleting agent of the present invention after a myocardial infarction.

In particular embodiments, the patient is administered with the depleting agent of the present invention simultaneously or sequentially (i.e. before or after) with the management of acute myocardial infarction. In particular embodiments, the patient is administered with the depleting agent of the present invention at the beginning of the management of acute myocardial infarction. In particular embodiments, the patient is administered with the depleting agent of the present invention after at least 1, 3, 5, 8, 12, 24, 36 or 48 hours of the management of acute myocardial infarction.

As used herein, the management of acute myocardial infarctions refers to the administration of the patients into a medical unit and the implementation of a revascularization procedure.

In some embodiments, the depleting agent of the present invention is administered simultaneously or sequentially (i.e. before or after) with a revascularization procedure performed on the patient. In some embodiments, the patient is administered with the depleting agent of the present invention before, during, and after a revascularization procedure. In some embodiments, the patient is administered with the depleting agent of the present invention as a bolus dose immediately prior to the revascularization procedure. In some embodiments, the patient is administered with the depleting agent of the present invention continuously during and after the revascularization procedure. In some embodiments, the patient is administered with the depleting agent of the present invention for a time period selected from the group consisting of at least 1 hours after a revascularization, at least 3 hours after a revascularization procedure; at least 5 hours after a revascularization procedure; at least 8 hours after a revascularization procedure; at least 12 hours after a revascularization procedure; at least 24 hours after a revascularization procedure, at least 36 hours after a revascularization procedure; at least 48 hours after a revascularization procedure. In some embodiments, the revascularization procedure is selected from the group consisting of percutaneous coronary intervention; balloon angioplasty; insertion of a bypass graft; insertion of a stent; directional coronary atherectomy; treatment with one or more thrombolytic agent(s); and removal of an occlusion.

As used herein, the term “NK cell”, also known as “Natural Killer cell”, has its general meaning in the art and refers to a sub-population of lymphocytes that is involved in non-conventional immunity. NK cells can be identified by virtue of certain characteristics and biological properties, such as the expression of specific surface antigens including CD56 and/or NKp46 for human NK cells, the absence of the alpha/beta or gamma/delta TCR complex on the cell surface, the ability to bind to and kill cells that fail to express “self” M1HC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. As used herein, the term NK cell does not include NKT cells.

As used herein, the term “depletion” with respect to NK cells, refers to a measurable decrease in the number of NK cells in the patient. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of NK cells in the patient below detectable limits.

In particular embodiment, the agent does not deplete NKT cells.

As used herein, the term “NKT cells”, also known as Natural Killer T cells”,”, has its general meaning in the art and refers to a sub-population of T cells whom are rapid responders of the innate immune system and mediate potent immunoregulatory and effector functions in a variety of disease settings. Like conventional T lymphocytes, NKT cells express a T cell receptor (TCR), which is generated by somatic DNA rearrangement. However, whereas the TCR repertoire of conventional T cells is highly diverse, most NKT cells, commonly referred to as invariant or type I NKT cells, express a semi-invariant TCR.

In some embodiments, the agent is an antibody having binding affinity for a NK receptor.

In some embodiments, the agent is an antibody having not binding affinity for a NKT receptor.

As used herein, the term “NK cell receptor” refers to any cell surface molecule that is found consistently on all or a fraction of NK cells. Preferably, the NK cell receptor is expressed exclusively on NK cells (resting or activated), although the term also encompasses receptors that are also expressed on other cell types. Examples of NK cell receptors include members of the KIR receptor family, CD94, NKG2 receptors, NCR receptors such as NKp30, NKp44, and NKp46, LIR-1, and others (see, e.g., Trowsdale and Parham (2004) Eur J Immunol 34(1):7-17; Yawata et al. (2002) Crit Rev Immunol 22(5-6):463-82; Hsu et al. (2002) Immunol Rev 190:40-52; Middleton et al. (2002) Transpl Immunol 10(2-3):147-64; Vilches et al. (2002) Annu Rev Immunol 20:217-51; OMIM 602894; Braud et al. (1998) Nature 391:795-799; Chang et al. (1995) Europ. J. Immun 25:2433-2437; Lazetic et al. (1996) Immun 157:4741-4745; Rodriguez et al. (1998) Immunogenetics 47:305-309; OMIM 161555; Houchins et al. (1991) J. Exp. Med. 173:1017-1020; Adamkiewicz et al. (1994) Immunogenetics 39:218; Renedo et al. (1997) Immunogenetics 46:307-311; Ravetch et al. (2000) Science 290:84-89; PCT WO 01/36630; Vitale et al. (1998) J. Exp. Med. 187:2065-2072; Sivori et al. (1997) J. Exp. Med. 186:1129-1136; Pessino et al. (1998) J. Exp. Med. 188:953-960; the disclosures of each of which is herein incorporated by reference).

In some embodiments, the agent is an antibody having binding affinity for a NK receptor selected from the group consisting of KIR2DL1, KIR2DS1, KIR2DL2, KIR2DL3, KIR2DS4, NKG2C, NKG2D, NKG2E, NKG2F, CD94, and NKG2A. In some embodiments, the agent is an antibody having binding affinity for a NK receptor selected from the group consisting of NKp30, NKp44, and NKp46.

In some embodiments, the antibody binds to NKG2A and binds substantially the same epitope as an antibody selected from the group consisting of Z199 and Z270. In some embodiments, the antibody binds KIR2DL1, KIR2DL2 and/or KIR2DL3 and binds substantially the same epitope as an antibody DF200, NKVSF or 1-7F9 described in PCT patent publication WO 2005/003172 and WO 06/003179, the disclosure of which are incorporated herein by reference.

In some embodiments, the antibody binds NKG2D and binds substantially the same epitope as an antibody selected from the group consisting of BAT221, ECM217, and ON72. In some embodiments, the antibody binds to NKp46 and binds substantially the same epitope as an antibody selected from the group consisting of BAT281. Anti-Nkp46 antibodies are well known in the art and typically includes those described in the International Patent Application WO2017114694 that is incorporated by reference. In some embodiments, the anti-Nkp46 antibody is the hNKp46.02 antibody that is disclosed in Berhani, Orit, et al. “Human anti-NKp46 antibody for studies of NKp46-dependent NK cell function and its applications for type 1 diabetes and cancer research.” European journal of immunology 49.2 (2019): 228-241. As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference).

Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein the term “bind” indicates that the antibody has affinity for the surface molecule. The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

In some embodiments, the the antibody is a fully human antibody, a humanized antibody or a chimeric antibody.

As used herein, the term “fully human” refers to an immunoglobulin, such as an antibody or antibody fragment, where the whole molecule is of human origin or consists of an amino acid sequence identical to a human form of the antibody or immunoglobulin. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used hereon, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

In some embodiments, the antibody suitable for depletion of NK cells mediates antibody-dependent cell-mediated cytotoxicity.

As used herein the term “antibody-dependent cell-mediated cytotoxicity” or “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. While not wishing to be limited to any particular mechanism of action, these cytotoxic cells that mediate ADCC generally express Fc receptors (FcRs).

As used herein, the term “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

As used herein, the terms “Fc receptor” or “FcR” are used to describe a receptor that binds to the Fc region of an antibody. The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells.

Alternatively, or additionally, ADCC activity of the molecules of interest may be assessed in vivo, e.g., in an animal model such as that disclosed in Clynes et al., Proc. Natl. Acad. Sci. (USA), 95:652-656 (1998).

As used herein, the term “effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

In some embodiments, the antibody suitable for depletion of NK cells is a full-length antibody. In some embodiments, the full-length antibody is an IgG1 antibody. In some embodiments, the full-length antibody is an IgG3 antibody.

In some embodiments, the antibody suitable for depletion of NK cells comprises a variant Fc region that has an increased affinity for FcγRIA, FcγRIIA, FcγRIIB, FcγRIIIA, FcγRIIIB, and FcγRIV. In some embodiments, the antibody of the present invention comprises a variant Fc region comprising at least one amino acid substitution, insertion or deletion wherein said at least one amino acid residue substitution, insertion or deletion results in an increased affinity for FcγRIA, FcγRIIA, FcγRIIB, FcγRIIIA, FcγRIIIB, and FcγRIV, In some embodiments, the antibody of the present invention comprises a variant Fc region comprising at least one amino acid substitution, insertion or deletion wherein said at least one amino acid residue is selected from the group consisting of: residue 239, 330, and 332, wherein amino acid residues are numbered following the EU index. In some embodiments, the antibody of the present invention comprises a variant Fc region comprising at least one amino acid substitution wherein said at least one amino acid substitution is selected from the group consisting of: S239D, A330L, A330Y, and 1332E, wherein amino acid residues are numbered following the EU index.

In some embodiments, the glycosylation of the antibody suitable for depletion of NK cells is modified. For example, an aglycosylated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site.

Such aglycosylation may increase the affinity of the antibody for antigen. Such an approach is described in further detail in U.S. Pat. Nos. 5,714,350 and 6,350,861 by Co et al. Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP 1176195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the human monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17: 176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the human monoclonal antibodies of the present invention can be produced in yeasts or filamentous fungi engineered for mammalian-like glycosylation pattern and capable of producing antibodies lacking fucose as glycosylation pattern (see for example EP1297172B1).

In some embodiments, the antibody suitable for depletion of NK cells mediated complement dependant cytotoxicity.

As used herein, the term “complement dependent cytotoxicity” or “CDC” refers to the ability of a molecule to initiate complement activation and lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed.

In some embodiments, the antibody suitable for depletion of NK cells mediates antibody-dependent phagocytosis.

As used herein, the term “antibody-dependent phagocytosis” or “opsonisation” refers to the cell-mediated reaction wherein nonspecific cytotoxic cells that express FcγRs recognize bound antibody on a target cell and subsequently cause phagocytosis of the target cell.

In some embodiments, the antibody suitable for depletion of NK cells is a multispecific antibody comprising a first antigen binding site directed against a NK receptor (e.g. Nkp46) and at least one second antigen binding site directed against an effector cell as above described. In said embodiments, the second antigen-binding site is used for recruiting a killing mechanism such as, for example, by binding an antigen on a human effector cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs. In some embodiments, the second binding site binds to a Fc receptor as above defined. Exemplary formats for the multispecific antibody molecules of the present invention include, but are not limited to (i) two antibodies cross-linked by chemical heteroconjugation, one with a specificity to a specific surface molecule of ILC and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivaient bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the multispecific antibody is thus a bispecific antibody.

In some embodiments, the bispecific antibody is a BiTE. As used herein, the term “Bispecific T-cell engager” or “BiTE” refers to a bispecific antibody that is a recombinant protein construct composed of two flexibly connected single-chain antibodies (scFv). One of said scFv antibodies binds specifically to a selected NK receptor, the second binds specifically to another molecule such as CD3, a subunit of the T-cell receptor complex on T cells. In some embodiments, the BiTE antibodies are capable of binding T cells transiently to target cells and, at the same time, activating the cytolytic activity of the T cells. The BiTE-mediated activation of the T cells requires neither specific T-cell receptors on the T cells, nor MHC I molecules, peptide antigens or co-stimulatory molecules on the target cell.

In some embodiments, the antibody suitable for depletion of NK cells is conjugated to a therapeutic moiety, i.e. a drug.

In some embodiments, the therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody suitable for depletion of NK cells is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof, an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof, an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody suitable for depletion of NK cells is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45(12): 3580-3584) and have anti-cancer (U.S. Pat. No. 5,663,149) and antifungal activity (Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the antibody suitable for depletion of NK cells is conjugated to pyrrolo[2,1-c][1,4]-benzodiazepine (PDB) or an analog, derivative or prodrug thereof. Suitable PDBs and PDB derivatives, and related technologies are described in, e.g., Hartley J. A. et al., Cancer Res 2010; 70(17): 6849-6858; Antonow D. et al., Cancer J 2008; 14(3): 154-169; Howard P. W. et al., Bioorg Med Chem Lett 2009; 19: 6463-6466 and Sagnou et al., Bioorg Med Chem Lett 2000; 10(18): 2083-2086.

In some embodiments, the antibody suitable for depletion of NK cells is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiments, the antibody suitable for depletion of NK cells is conjugated to an anthracycline or an analog, derivative or prodrug thereof. In some embodiments, the antibody is conjugated to maytansine or an analog, derivative or prodrug thereof. In some embodiments, the antibody is conjugated to calicheamicin or an analog, derivative or prodrug thereof. In some embodiments, the antibody is conjugated to duocarmycin or an analog, derivative or prodrug thereof. In some embodiments, the antibody is conjugated to rachelmycin (CC-1065) or an analog, derivative or prodrug thereof. In some embodiments, the antibody is conjugated to dolastatin 10 or an analog, derivative or prodrug thereof. In some embodiments, the antibody is conjugated to dolastatin 15 or an analog, derivative or prodrug thereof. In some embodiments, the antibody is conjugated to monomethyl auristatin E or an analog, derivative or prodrug thereof. In some embodiments, the antibody is conjugated to monomethyl auristatin F or an analog, derivative or prodrug thereof. In some embodiments, the antibody is conjugated to pyrrolo[2,1-c][1,4]-benzodiazepine or an analog, derivative or prodrug thereof. In some embodiments, the antibody is conjugated to irinotecan or an analog, derivative or prodrug thereof.

In some embodiments, the antibody suitable for depletion of NK cells is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

Techniques for conjugating molecule to antibodies, are well-known in the art (See, e.g., Amon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

As used herein, the term “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the active ingredient of the present invention (e.g. depleting agent of the present invention) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1: Analysis of CD45+CD3-CD19-Ly6G-NK1.1+NK infiltration in the heart at different time point in MI and sham-operated C57B16 mice by flow cytometry. N=4-5/group*P<0.001

FIG. 2: Analysis by ScRNA sequencing of mRNA expression (Gzm for Granzym and Perf for perforin) in different cell types in the ischemic heart at day 5 after MI.

FIG. 3: Heart tissue necrosis was staining using TTC at day 3 and quantified along heart tissue section. We found a significant reduction of Heart tissue necrosis in NK deficient mice. N=4/group, *, P<0.05.

FIG. 4: Infarct was staining using Masson trichrome at day 21 after myocardial infarction and quantified along heart tissue section. We found a significant reduction of infarct size in NK deficient mice. N=4-5/group, *, P<0.05.

FIG. 5: At day 21, left ventricular systolic function was quantified by echocardiography. We found a significant higher left ventricular ejection fraction in NK deficient mice. N=4-5/group, *, P<0.05.

FIG. 6: Kinetic of NK cells in the blood after anti-NK1.1 depleting antibody injection. Anti-NK1.1 mAb induced a rapid, almost full and sustained NK cell depletion (Red). N=5/group, ** P<0.01.

FIG. 7: Analysis of Illb, 116 and Il-10 mRNA expression in the ischemic heart tissue by qPCR at day 7 after MI in control and NK depleted mice. NK cell depletion mitigates pro-inflammatory responses in the ischemic heart tissue. N=5-10/group, *, P<0.05, **, P<0.01.

FIG. 8: Infarct was staining using Masson trichrome at day 21 after myocardial infarction and quantified along heart tissue section. We found a significant reduction of infarct size in NK depleted mice. N=6-8/group.

FIG. 9: At day 21, left ventricular systolic function was quantified by echocardiography. We found a significant higher left ventricular ejection fraction in NK depleted mice. N=5-7/group, *, P<0.05.

FIG. 10: C57B16 mice were treated either with PBS or anti-NK1.1 depleting antibody IP 1 hour after MI. A, at day 3, heart tissue necrosis was quantified after TTC (Triphenyltetrazolium chloride) staining (N=5/group). B, Granzyme B mRNA levels were quantified by qPCR in the heart tissue in PBS- and NK1.1-treated mice at day 5 (N=9-10/group). *, p<0.05 non parametric test.

### EXAMPLE

### 1) NK Cells are Recruited in the Ischemic Heart Tissue in Mouse

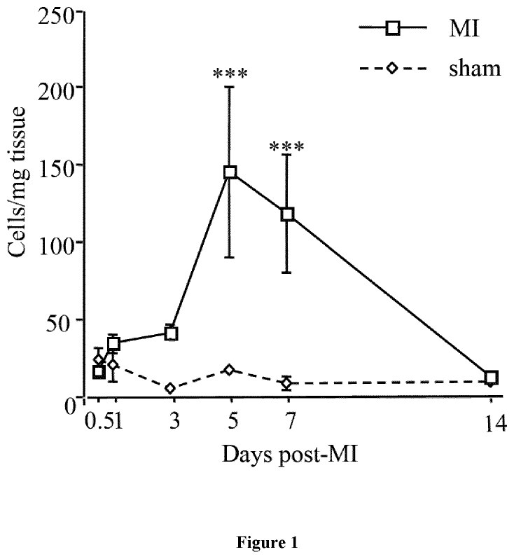

Using a model of permanent coronary artery ligation in mice, we analyzed NK cell infiltration in the heart at different time point using flow cytometry after heart tissue enzymatic digestion. We found that NK cell number in the heart was very low before MI, but markedly increased at day 1 and peaked at day 5 after coronary ligation (FIG. 1).

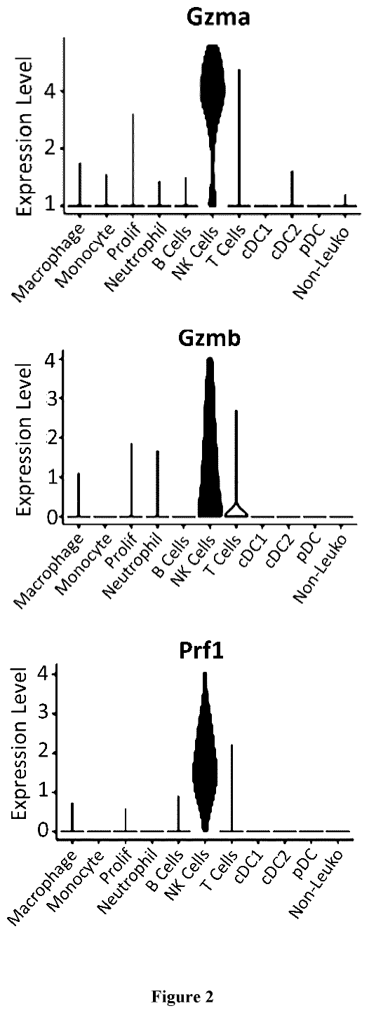

NK cells infiltration in the ischemic heart tissue has been confirmed by immunostaining. Sc RNA sequencing showed that infiltrating NK cells express cytotoxic mediators such as Perforin and Granzyme A/B at day 5 after MI (FIG. 2).

### 2) NK Cells are Detected in Human Ischemic Heart Tissue

Human heart tissue were obtained from patients with left ventricle assist device (HeartMate II or HeartWare) for ischemic cardiogenic shock. For implantation of the device the apex of the left ventricle was open and tissue samples were excised. Tissues were fixed in formalin and paraffin embedded. Immunohistochemistry stainings showed NKp46+NK cells in the ischemic heart tissue around necrotic areas (not shown).

### 3) NK Deficiency Protects Against Deleterious Post-Ischemic Cardiac Remodeling

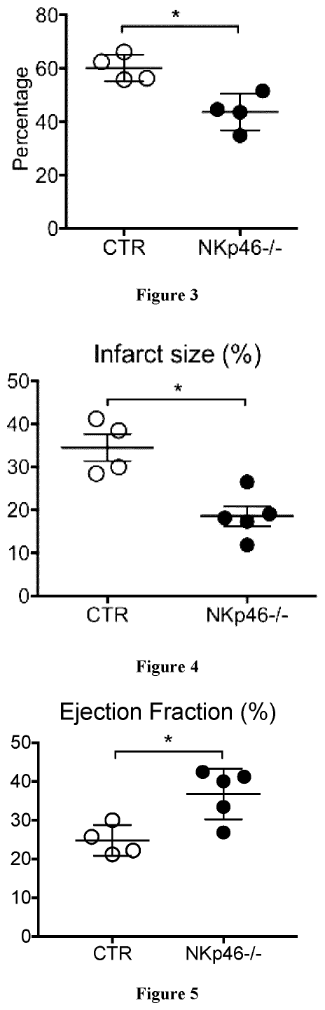

To evaluate the role of NK cells in post-ischemic cardiac remodeling, we used NKp46^{iCre} R26R^{DTA} (called NKp46^{-/-}) mice, in which NK cells are selectively depleted [14]. At day 3, we confirmed the absence of NK cells in NKp46^{-/-} mice in the spleen as well as in the heart tissue (not shown). After permanent coronary occlusion, we found a significant reduction of heart tissue necrosis at day 3 in NK deficient animals (−25%, FIG. 3) when compared to control animals and a significant reduction of infarct size at day 21 (FIG. 4). At Day 21, we found a significant higher left ventricular systolic function in NK deficient (FIG. 5).

Overall, we showed here for the first time that NK cells play a pathological role in post-ischemic cardiac remodeling and depleting NK cells may be a promising therapeutic treatment in the future to limit ischemic heart failure.

### 4) NK Cells Depletion Attenuates Heart Pro-Inflammatory Signature and Protects Against Deleterious Post-Ischemic Cardiac Remodeling

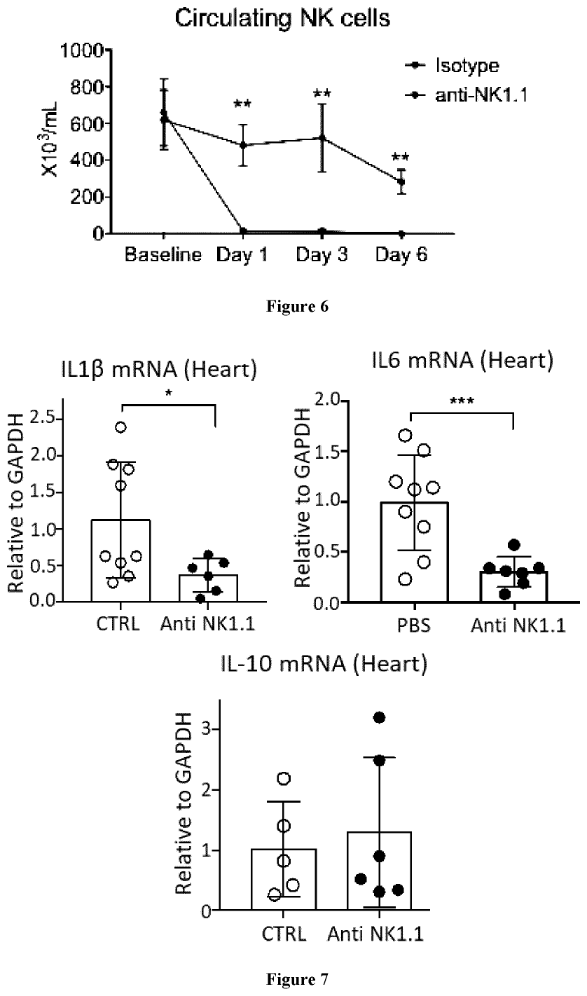

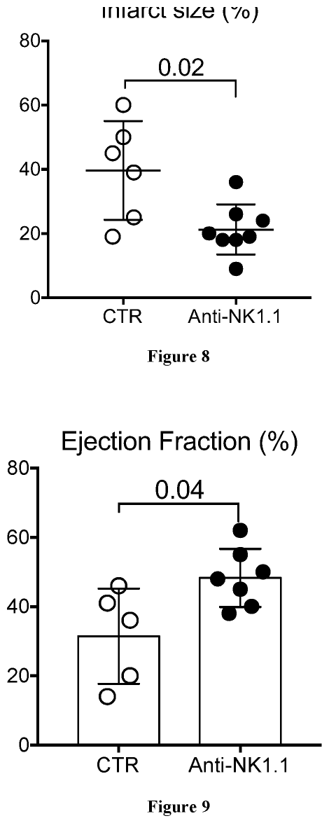

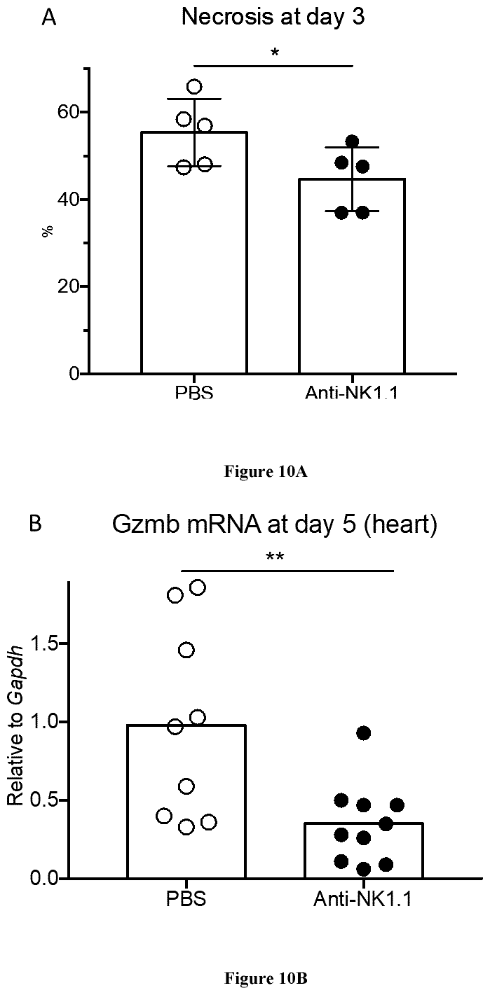

Next, we evaluated a therapeutic strategy based on NK depletion using anti-NK1.1 depleting antibody (clone PK136). NK1.1 is mainly expressed by NK cells and also by NKT cells which are detected in very low number in the heart following myocardial infarction. Anti-NK1.1 mAb (clone PK136) was injected IP one hour after MI with an additional injection at day 7. Anti-NK1.1 mAb led to a rapid and full NK cell depletion (FIG. 6). NK cell depletion mitigated pro-inflammatory response in the heart with a significant decrease of Il1b and Il6 mRNA expression in the ischemic heart tissue at day 7 (FIG. 7). At day 21, we found that NK depletion was protective with a significant decrease in infarct size (FIG. 8) and a better left ventricular systolic function (FIG. 9). We also found that NK cell depletion induces a major decrease in myocardial Granzyme B mRNA levels, a cytotoxic player, and a decrease in myocardial necrosis at D3 as measured by TTC (FIGS. 10A and 10B). The protective effect of NK depletion could thus related to a decrease in the cytotoxicity of NK cells on the myocardium via a local release of Granzyme B.

### CONCLUSION

In the context of acute myocardial infarction, NK cells are recruited in the ischemic heart tissue. NK deficiency protects against deleterious cardiac remodeling following myocardial infarction. NK depletion using monoclonal antibody attenuates pro-inflammatory response in the heart, protects against deleterious post-ischemic cardiac remodeling and represents a promising strategy to limit ischemic heart failure.

### REFERENCES

Throughout this application, various references describe the state of the art to which this invention pertains. The disclosures of these references are hereby incorporated by reference into the present disclosure.
1. Mortality, G. B. D. and C. Causes of Death, Global, regional, and national age-sex specific all-cause and cause-specific mortality for 240 causes of death, 1990-2013: a systematic analysis for the Global Burden of Disease Study 2013. Lancet, 2015. 385(9963): p. 117-71.
2. Frangogiannis, N. G., The inflammatory response in myocardial injury, repair, and remodeling. Nat Rev Cardiol, 2014. 11(5): p. 255-65.
3. Puymirat, E., et al., Association of changes in clinical characteristics and management with improvement in survival among patients with ST-elevation myocardial infarction. JAMA, 2012. 308(10): p. 998-1006.
4. Lavoie, L., et al., Burden and Prevention of Adverse Cardiac Events in Patients with Concomitant Chronic Heart Failure and Coronary Artery Disease: A Literature Review.

Cardiovasc Ther, 2016. 34(3): p. 152-60.
5. Ridker, P. M., et al., Antiinflammatory Therapy with Canakinumab for Atherosclerotic Disease. N Engl J Med, 2017.
6. Spits, H., et al., Innate lymphoid cells—a proposal for uniform nomenclature. Nat Rev Immunol, 2013. 13(2): p. 145-9.
7. Bordon, Y., Mucosal immunology: ILCs broker peace deals in the gut. Nat Rev Immunol, 2013. 13(7): p. 473.
8. Vosshenrich, C. A. and J. P. Di Santo, Developmental programming of natural killer and innate lymphoid cells. Curr Opin Immunol, 2013. 25(2): p. 130-8.
9. Vivier, E., et al., Innate Lymphoid Cells: 10 Years On. Cell, 2018. 174(5): p. 1054-1066.
10. Shmeleva, E. V., et al., Differences in immune responses between CMV-seronegative and -seropositive patients with myocardial ischemia and reperfusion. Immun Inflamm Dis, 2015. 3(2): p. 56-70.
11. Yan, X., et al., Temporal dynamics of cardiac immune cell accumulation following acute myocardial infarction. J Mol Cell Cardiol, 2013. 62: p. 24-35.
12. Gan, Y., et al., Ischemic neurons recruit natural killer cells that accelerate brain infarction. Proc Natl Acad Sci USA, 2014. 111(7): p. 2704-9.
13. Fahrner, R., et al., Tumor necrosis factor-related apoptosis-inducing ligand on NK cells protects from hepatic ischemia-reperfusion injury. Transplantation, 2014. 97(11): p. 1102-9.
14. Deauvieau, F., et al., Lessons from NK Cell Deficiencies in the Mouse. Curr Top Microbiol Immunol, 2016. 395: p. 173-90.

1. A method of treating adverse post-ischemic cardiac remodeling in a patient who experienced a myocardial infarction comprising administering to the patient a therapeutically effective amount of an agent that depletes NK cells.

2. A method for preventing heart failure in a patient who experienced a myocardial infarction comprising administering to the patient a therapeutically effective amount of an agent that depletes NK cells.

3. The method of claim 1 wherein the depleting agent is administered simultaneously or sequentially with a revascularization procedure performed on the patient.

4. The method of claim 1 wherein the agent is an antibody having binding affinity for a NK receptor.

5. The method of claim 4 wherein the agent is an antibody having binding affinity for a NK receptor selected from the group consisting of KIR2DL1, KIR2DS1, KIR2DL2, KIR2DL3, KIR2DS4, NKG2C, NKG2D, NKG2E, NKG2F, CD94, and NKG2A.

6. The method of claim 4 wherein the agent is an antibody having binding affinity for a NK receptor selected from the group consisting of NKp30, NKp44, and NKp46.

7. The method of claim 6 wherein the agent is an antibody having binding affinity for NKp46.

8. The method of claim 4 wherein the antibody mediates antibody-dependent cell-mediated cytotoxicity.

9. The method of claim 4 wherein the antibody is a multispecific antibody comprising a first antigen binding site directed against a NK receptor and at least one second antigen binding site directed against an effector cell.

10. The method of claim 4 wherein the antibody is conjugated to a cytotoxic moiety.

11. The method of claim 2 wherein the depleting agent is administered simultaneously or sequentially with a revascularization procedure performed on the patient.

12. The method of claim 2 wherein the agent is an antibody having binding affinity for a NK receptor.

13. The method of claim 12 wherein the agent is an antibody having binding affinity for a NK receptor selected from the group consisting of KIR2DL1, KIR2DS1, KIR2DL2, KIR2DL3, KIR2DS4, NKG2C, NKG2D, NKG2E, NKG2F, CD94, and NKG2A.

14. The method of claim 12 wherein the agent is an antibody having binding affinity for a NK receptor selected from the group consisting of NKp30, NKp44, and NKp46.

15. The method of claim 14 wherein the agent is an antibody having binding affinity for NKp46.

16. The method of claim 12 wherein the antibody mediates antibody-dependent cell-mediated cytotoxicity.

17. The method of claim 12 wherein the antibody is a multispecific antibody comprising a first antigen binding site directed against a NK receptor and at least one second antigen binding site directed against an effector cell.

18. The method of claim 12 wherein the antibody is conjugated to a cytotoxic moiety.
